(19) United States
(12) Patent Application Publication
(10) Pub. No.: US 2010/0034905 A1
(43) Pub. Date: Feb. 11, 2010

(54) Method of treating burns

(76) Inventor: Ruben Sembrano, Hasbrouck, NJ (US)

Correspondence Address: KLAUBER & JACKSON, 411 HACKENSACK AVENUE, HACKENSACK, NJ, 07601, US

(21) Appl. No.: 12/462,518

(22) Filed: Aug. 5, 2009

Related U.S. Application Data
(60) Provisional application No. 61/137,919, filed on Aug. 5, 2008.

(51) Int. Cl. A61K 36/746

(52) U.S. Cl. 424/725

(57) ABSTRACT

In a first aspect, the present invention provides compositions for treating burns. The compositions, containing effective amounts of a processed Morinda citrifolia product may be used to stimulate, expedite or assist in healing the burned skin of patients. The processed Morinda citrifolia product may be a fruit juice, a puree juice, a fruit or puree juice concentrate, an oil extract, dietary fiber, or an alcohol or aqueous extract. The compositions may be formulated into creams, lotions, gels, aerosols and the like. Other optional ingredients may be added including, for instance, fillers, emulsifiers, thickeners, emollients, fragrances and dyes. The present invention further provides a method for treating a burn injury by administering a topical composition comprising at least one processed Morinda citrifolia product.

### BACKGROUND OF THE INVENTION

A wound is a defect or break in the skin that comes from physical, mechanical or thermal damage including burns. There are several different types of burns including thermal, chemical, electrical, and radiation induced burns. Bums may be classified according to degree such as deep dermal (second degree) and full thickness (third degree) burns where most of the protective epithelium layer of the skin is destroyed. Such full thickness burns can become infected with bacteria or yeast. Infection in a burn wound is the most common management problem for burn patients. Such infections are life threatening as they may develop into a septicemia.

The use of skin treatment compositions for treating a variety of skin disorders is well known in the prior art. Some known skin treatment compositions include, for instance, those described in U.S. Pat. No. 4,276,281; U.S. Pat. No. 4,839,159; U.S. Pat. No. 4,837,019; U.S. Pat. No. 5,166,132; U.S. Pat. No. 5,382,431; and U.S. Pat. No. 4,393,045; U.S. Pat. No. 5,766,614 and U.S. Pat. No. 7,014,873. However, none of these provide novel compositions for burn treatment.

The Indian Mulberry or Noni plant, Morinda citrifolia L. (“Morinda citrifolia”), is a shrub or small tree that grows up to 10 m in height having leaves arranged elliptically. Its small white flowers are contained in a fleshy, head-like cluster. The fruits are large, fleshy, and ovoid. The fruits are creamy-white and edible, but they have an unpleasant taste and odor. The plant is a native of Southeast Asia and has spread to a large area from India to eastern Polynesia. It grows randomly in the wild, and it has been cultivated. The Morinda citrifolia flowers are small, white, three to five lobed, tubular, fragrant, and about 1.25 cm long. The flowers develop into compound fruits of many small drupes fused into an ovoid, ellipsoid or roundish, lumpy body, with waxy, white, or greenish-white or yellowish, semi-translucent skin. The fruit has eyes on its surface similar to a potato. The fruit is juicy, bitter, dull-yellow or yellow-white, and contains numerous red-brown, hard, oblong or triangular, 2-celled stones, each containing four seeds.

The fruit has an offensive odor. Although the fruit has been eaten by several nationalities as food, the most common use of Morinda citrifolia is as a source for red and yellow dye. There has been an increasing interest in the nutritional and health benefits of the Morinda citrifolia plant. Because its fruit is inedible to most, the fruit must be processed in order to make it suitable for human consumption.

Processed Morinda citrifolia fruit juice may be prepared by separating seeds and peels from the juice and pulp of a ripened Morinda citrifolia fruit and filtering the pulp from the juice. The juice can also be immediately included as an ingredient in another food product, frozen or pasteurized. Also, the juice and pulp can be pureed into a homogenous blend to be mixed with other ingredients. Likewise, the fruit and juice may be freeze dried. The fruit and juice can be reconstituted during production of the final juice product.

Morinda citrifolia has been shown to have relatively high amounts of proxeronine, proxeronase, and xeronine. Proxeronase is an enzyme and proxeronine is the molecule that is cleaved by the proxeronase to form the alkaloid xeronine. Xeronine is an essential component of the protein in cell wall membranes. It is incorporated into cell walls upon synthesis. Morinda citrifolia also is relatively high in damnacanthal, a chemical reportedly possessing cancer fighting abilities at least in part by stimulating T cells or killer T cell activity. Further, Morinda citrifolia is relatively high in scopoletin which has been shown in laboratory tests to have analgesic properties, to demonstrate serotonin binding, and to have potential anti-cancer effects. Still further, Morinda citrifolia is relatively high in terpenes which may demonstrate some cell rejuvenation properties along with being relatively rich in glycosides, amino acids, fiber and essential fatty acids.

### SUMMARY OF THE INVENTION

In a first aspect, the present invention provides compositions for treating burns. The compositions may be used to stimulate, expedite or assist in healing the burned skin of patients. The compositions of the present invention contain effective amounts of one or more processed Morinda citrifolia products. The processed Morinda citrifolia product may be a fruit juice, a puree juice, a fruit or puree juice concentrate, an oil extract, dietary fiber, or an alcohol or aqueous extract. The present compositions may be formulated into creams, lotions, gels, aerosols and the like. Other optional ingredients may be added including, for instance, fillers, emulsifiers, thickeners, emollients, fragrances and dyes. The compositions are preferably suitable for topical administration, and the compositions may contain various amounts and concentrations of the processed Morinda citrifolia product, along with other various ingredients, such as a carrier medium or composition in a topical dermal formulation.

In preferred embodiments, the processed Morinda citrifolia product may comprise one or more, for example, two, three, four or five of processed Morinda citrifolia fruit juice (in dilute or concentrate form), processed Morinda citrifolia puree juice (in dilute or concentrate form), processed Morinda citrifolia dietary fiber, and processed Morinda citrifolia oil or oil extract. Moreover, this composition may comprise extracted Morinda citrifolia products, such as Morinda citrifolia ethyl extracts, Morinda citrifolia ethanol extracts, and Morinda citrifolia methanol extracts. The processed Morinda citrifolia fruit juice (in dilute or concentrate form) may be present in an amount of at least 1%, 5%, 10%, 25%, 50%, 75% or 90% or more by weight. The processed Morinda citrifolia puree juice (in dilute or concentrate form) may be present in an amount of at least 1%, 5%, 10%, 25%, 50%, 75% or 90% or more by weight. The processed Morinda citrifolia dietary fiber may be present in an amount of at least 1%, 5%, 10%, 25%, 50%, 75% or 90% or more by weight. The processed Morinda citrifolia oil or oil extract may be nresent in an amount of at least 1%, 5%, 10%, 25%, 50%, 75% or 90% or more by weight. Moreover, this composition may comprise extracted Morinda citrifolia products, such as Morinda citrifolia ethyl extracts, Morinda citrifolia ethanol extracts, and Morinda citrifolia methanol extracts which may each be present in an amount of at least 1%, 2%, 5%, 10%, 20%, 25%, 30%, 40%, 50%, 75% or 90% or more by weight. In some embodiments, the processed Morinda citrifolia product may comprise one, two, three, four or all five of the following ingredients: Morinda citrifolia fruit juice (in dilute or concentrate form), processed Morinda citrifolia puree juice (in dilute or concentrate form), processed Morinda citrifolia dietary fiber, and processed Morinda citrifolia oil or oil extract. Moreover, in some embodiments the composition may comprise one, two or all three of the following extracted Morinda citrifolia products Morinda citrifolia ethyl extracts, Morinda citrifolia ethanol extracts, and Morinda citrifolia methanol extracts.

In a second aspect, the present invention features a method for treating a burn injury by administering a topical composition comprising at least one processed Morinda citrifolia product. The processed Morinda citrifolia product may be a fruit juice, a puree juice, a fruit or puree juice concentrate, an oil extract, dietary fiber, or an alcohol or aqueous extract. Morinda citrifolia can be processed and used within a formulation to treat, stimulate the healing of or alleviate the symptoms of a burn wound. The method may stimulate, expedite or assist in healing the burned skin of patients.

### DETAILED DESCRIPTION OF THE INVENTION

Morinda citrifolia and the Methods Used to Produce Processed Morinda citrifolia Products. The present compositions contain one or more of fruit juice, puree, and oil extracted from the Morinda Citrifolia plant. The fruit is either picked by hand or by mechanical equipment to produce Morinda citrifolia fruit juice. The fruit may be harvested when it is at least one inch (2-3 cm) up to 12 inches (24-36 cm) in diameter. The fruit preferably ranges from a dark green, a yellow-green or a white color. The fruit is cleaned after harvesting before any processing. The fruit may be allowed to ripen or age from 0 to 14 days, with most fruit being ripened for from 2 to 3 days, by being placed on equipment to avoid contact with the ground. The fruit may be covered with a cloth or netting material during aging or be aged without being covered. The fruit is allowed to become a light color, from light green, light yellow, white to translucent before processed further. The fruit may be inspected for spoilage or for excessively green color adlu firmness. Spoiled and hard green fruit may be separated from the acceptable fruit.

The ripened and aged fruit is placed in suitable containers for further processing and transport. The containers of aged fruit may be held from 0 to 30 days, preferably 7 to 14 days before processing. The containers can sometimes be refrigerated prior to further processing. The fruit is unpacked from the storage containers and processed through a manual or mechanical separator. The seeds and peel are separated from the juice and pulp. The juice and pulp may be packaged into suitable containers for storage and transport or the juice and pulp may be immediately processed into a finished juice product. The product may be stored in refrigerated, frozen, or room temperature conditions. The juice and pulp may be blended into a homogenous blend. They may be mixed with other ingredients, such as flavorings, sweeteners, nutritional ingredients, botanicals, and colorings. The finished juice product is preferably heated and pasteurized at a minimum temperature of 181° F. up to 212° F. Morinda citrifolia puree and puree juice may also be produced in either concentrate or diluted form. Puree is the pulp as separated from the seeds.

The juice and pulp may be further processed by separating the pulp from the juice through filtering equipment that may include a centrifuge decanter, a screen filter, a filter press, reverse osmosis filtration, or other standard commercial filtration devices. The wet pulp may also be pasteurized at a temperature of 181° F. or above. Drying may further process the wet pulp by freeze-drying, drum drying, tray drying, sun drying, or spray drying. The dried Morinda citrifolia pulp may have a moisture content of from 0.1 to 15 percent by weight, preferably from 5 to 10 percent by weight and have a fiber content of 0.1 to 25 percent by weight, preferably from 5 to 15 percent by weight.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Some typical fiber products include guar gum, gum arabic, soybean fiber, oat fiber, pea fiber, fig fiber, citrus pulp sacs, hydroxymethylcellulose, cellulose, seaweed, food grade lumber or wood pulp, and hemicellulose. Other supplemental fiber ingredients may come from grains or grain products and be present in amounts of 0 up to 30 percent, by weight, preferably 10 to 30 percent by weight.

Sweeteners may include, for instance, natural sugars corn, sugar beet, sugar cane, potato, tapioca, or other starch-containing sources that can be chemically or enzymatically converted to crystalline chunks, powders, and/or syrups. Also sweeteners may include artificial or high intensity sweeteners such as sucralose, stevia, and saccharin. The concentration of sweeteners may be 0 to 50 percent by weight, preferably about 1 and 5 percent by weight.

Flavors may include artificial or natural flavor or ingredients. The concentration of flavors may be from 0 to 15 percent by weight, preferably 1 to 5 percent. Colors may include food grade artificial or natural coloring agents from 0 to 10 percent by weight.

Nutritional ingredients may include vitamins, minerals, trace elements, herbs, botanical extracts, bioactive chemicals and compounds at 0 to 10 percent by weight, preferably 1 to 5 percent by weight. Vitamins that may be present in the composition include, for instance, vitamins A, B1 through B12, C, D, E, Folic Acid, Pantothenic Acid, and Biotin. Examples of minerals and trace elements in the composition may include, for instance, one or more of calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Shitake mushroom, spirulina seaweed, grape seed extract, etc. Bioactive chemicals may include, for instance, caffeine, ephedrine, L-camitine, creatine, lycopene, etc.

The juice and pulp may be dried by one of a variety of methods, and the juice and pulp mixture may be pasteurized or enzymatically treated prior to drying. The product may be heated to a temperature between 75° F. and 135° F. and then treated with either a single enzyme or a combination of enzymes. These enzymes include, for instance, amylase, lipase, protease, cellulase, and bromelin. The juice and pulp may also be dried with other ingredients. The dried juice and pulp may be 1 to 20 percent moisture, 0.1 to 15 percent protein, and 0.1 to 20 percent. The filtered juice and the water from washing the wet pulp are preferably mixed together. The filtered juice may be vacuum evaporated to a moisture of 0.1 to 80 percent, preferably 20 to 75 percent. The resulting concentrated Morinda citrifolia juice may or may not be pasteurized.

The processed Morinda citrifolia product may also be a dietary fiber produced from the fruit puree. Moreover, the processed Morinda citrifolia product may also be in oil form, such as an oil extract. The Morinda citrifolia oil typically includes a mixture of several different fatty acids as triglycerides, such as palmitic, stearic, oleic, and linoleic fatty acids, and other fatty acids present in lesser quantities. Furthermore, the oil preferably includes an antioxidant such as a food grade antioxidant well known in the art.

The Morinda citrifolia plant is rich in natural ingredients such as alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl)benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chiororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid.

Many health benefits have been discovered from using products containing Morinda citrifolia. One benefit of Morinda citrifolia is its ability to isolate and produce xeronine. Xeronine occurs in practically all healthy cells of plants, animals and microorganisms. Morinda citrifolia contains significant amounts of the precursor of xeronine, proxeronine. Further, Morinda citrifolia contains the inactive form of the enzyme proxeronase which releases xeronine from proxeronine.

Formulations and Methods of Application. The present compositions are preferably suitable for topical application or administration as, for example, a lotion, gel, ointment, cream, oral medication, suppository, or others as commonly known in the art. The compositions containing a processed Morinda citrifolia product intended for topical application may be prepared according to any method known in the art for the manufacture of such topical compositions and may comprise any known additional ingredients commonly used to produce topical dermal products. Moreover, the processed Morinda citrifolia products may be combined with various other ingredients commonly used to treat burn wounds.

The compositions of the present invention comprise one, two, three, four, five or more processed Morinda citrifolia product present in an amount by weight from 0.01 to 100 percent by weight, preferably between 0.01 and 95 percent by weight, more preferably 1% to 90%, especially 5% to 85%. The processed Morinda citrifolia product is present as an active ingredient either alone or combination with one or more other active ingredients. The composition is useful for stimulating healing of burn wounds, or for expediting the healing

Optional Ingredients. Optional ingredients may be provided to the compositions. These ingredients are normally provided in amounts that do not alter the desired properties of the compositions. Ingredients that may be added may allow the compositions to be used in different forms, such as creams, gels, lotions and so forth. These ingredients include fillers such as chalk, magnesium oxide and carbonate, clay, talc, fused silica, and mixtures thereof. Thickeners may include natural and synthetic types. The thickeners used can include but are not limited to xanthan, karaya, guar gum, clay tragacanth various cellulostic materials such as starches.

The topical composition may be applied directly to the burn area as often as needed until the burn wound is completely healed. The compositions may be administered to any region of the body having a burn wound. The composition, as a topical, may be applied to the skin or mucosa of the patient, and may be absorbed or internalized into the body through the pores or membrane. Several embodiments of the topical composition having various different ingredients are contemplated with each embodiment having one or more forms of a processed Morinda citrifolia product as the active ingredient, optionally along with one or more carrier agents or mediums and any other ingredients commonly known in the art that are appropriate for a topical composition. In some embodiments, the topical composition comprises a processed Morinda citrifolia product present in an amount by weight between about 10 to 80 percent; and a carrier medium or composition present in an amount by weight between about 20 to 90 percent.

The processed Morinda citrifolia product may comprise one or more of processed Morinda citrifolia fruit juice (in dilute or concentrate form), processed Morinda citrifolia puree juice (in dilute or concentrate form), processed Morinda citrifolia dietary fiber, and processed Morinda citrifolia oil or oil extract. Moreover, this composition may comprise extracted Morinda citrifolia products, such as Morinda citrifolia ethyl extracts, Morinda citrifolia ethanol extracts, Morinda citrifolia methanol extracts, etc.

In another embodiment, the composition comprises processed Morinda citrifolia fruit juice or puree juice (in dilute or concentrate form) present in an amount by weight between about 0.1 to 80 percent; processed Morinda citrifolia oil present in an amount by weight between about 0.1 to 20 percent; and a carrier medium or composition present in an amount by weight between about 20 to 90 percent. The topical dermal composition may also be formulated with a Morinda citrifolia dietary fiber product in similar concentrations.

The carrier medium or composition in the topical dermal composition may comprise any ingredient capable of being safely introduced into the body of a mammal, and that is also capable of providing the carrying medium for the processed Morinda citrifolia product. Specific carrier mediums and compositions are well known in the art and not described in detail herein. The following non-limiting examples, which include the best mode, are set forth to illustrate the present invention.

The compositions may be administered directed to the skin or burn wound or they may be administered in a bandage or wrap to the skin or burn wound. They may be administered daily, hourly, one, two, three, four, five, six or more times a day, or they may be administered one, two, three, four or more times a week. Those of skill in the art may readily determine the treatment regimen depending upon the concentration of active ingredient and upon the severity and size of the burn wound. The compositions may be administered alone or in combination with other topical compositions either concurrently or sequentially.

### Examples

### Example 1

A composition according to the present invention will be applied on the skin of patients suffering I, II, and III degree burns. Upon application of a bandage containing a composition according to the present invention, one or more of the following observations will be noted: arrest of bleeding, reduction of pain, stoppage of purulent discharge, acceleration of granulation and epithelization. There will be exceedingly few cases of treatment complication, such as suppuration, bleeding, occurrence of a thick coat on the surface, prolongation of wound healing, etc. The composition according to the present invention will demonstrate a marked effect in the treatment of III degree burn, when all the sources for epithelium reprouuction in the injured area needed for skin regeneration are destroyed.

1. A topical composition for treating a burn wound comprising a processed Morinda citrifolia product.

2. A topical composition according to claim 1 wherein the processed Morinda citrifolia product is selected from the group consisting of a fruit juice, a puree juice, a fruit or puree juice concentrate, an oil extract, dietary fiber, or an alcohol or an aqueous extract.

3. A topical composition according to claim 1 wherein the processed Morinda citrifolia product is formulated into one selected from the group consisting of a cream, a lotion, a gel, and an aerosol.

4. A topical composition according to claim 1 further comprising one or more selected from the group consisting of a filler, an emulsifier, a thickener, an emollient, a fragrance and a dye.

5. A method for treating a burn injury by administering a topical composition according to claim 1.
